(19) United States
(12) Patent Application Publication
(10) Pub. No.: US 2015/0359818 A1
(43) Pub. Date: Dec. 17, 2015

(54) METHOD FOR PRODUCING A GAS TRANSPORTING RHEOLOGICAL MEDIUM

(71) Applicant: C. Edward Eckert, New Kensington, PA (US)

(72) Inventor: C. Edward Eckert, New Kensington, PA (US)

(21) Appl. No.: 14/489,681

(22) Filed: Sep. 18, 2014

Related U.S. Application Data
(63) Continuation-in-part of application No. 14/302,395, filed on Jun. 11, 2014.

(51) Int. Cl. A61K 33/00; A61L 15/26; A61L 15/18; A61L 15/60

(52) U.S. Cl. CPC A61K 33/00; A61L 15/60; A61L 15/26; A61L 15/18; A61L 2300/10; A61L 2300/412

(57) ABSTRACT

A method for producing a gas transporting rheological medium, said method comprises: (a) hydrating and dispersing a thickening agent in a fluid medium; (b) adding a first gas for dissolution and/or adsorption the fluid medium using a minimum fluid system pressure of about 7 psig; (c) mechanically emulsifying dimethylpolysiloxane into the fluid medium; (d) adding a source of one or more +2 valence cations to the suspension for 10 crosslinking to form a gel; and (e) dispensing the gel into a container.

### CROSS-REFERENCE TO RELATED APPLICATIONS

This application is a continuation-in-part of U.S. application Ser. No. 14/302,395, filed on Jun. 11, 2014, which itself claimed priority back to U.S. Provisional Application Ser. No. 61/833,857, filed on Jun. 11, 2013, both disclosures of which are fully incorporated by reference herein.

### BACKGROUND OF THE INVENTION

1. Technical Field of the Invention

This invention relates to dissolved gas media comprising one or more gas solutes and one or more solvents. More specifically, the invention relates to a medically useful media comprised of a liquid, lotion, and/or gel matrix having additional gas adsorption into one or more dispersed phase(s).

2. Description of the Related Art

In the medical and veterinary community, the effect of oxygen on living tissue is generally characterized by one of three regimes: metabolic enhancement (growth acceleration), metabolic inhibition (growth arrest), and toxicity. Oxygenated solutions have been used to accelerate the healing and regeneration rate of damaged tissue for wounds such as cuts, lacerations, sores and burns on the body. When these wounds begin to heal, fibroblastic cells divide and spread throughout the wound area. These fibroblastic cells produce collagen, an important protein that facilitates healing. Supplying sufficient quantities of oxygen to the wound area significantly enhances fibroblast proliferation.

Oxygen and oxidizing gases may also prove useful for their effect on biofilms, which are colonizations of microorganisms that adhere to one another and to a substrate. When colonized on living tissue, these films are believed to be highly detrimental to the tissue healing process because they can inhibit the exchange of oxygen and access to the wound site by pharmaceutical agents such as antibiotics. Oxygen and other oxidizing species may be expected to mediate a biofilm through an oxidative process.

In addition to treating wounds, oxygen is used in topical applications for cleaning and revitalizing skin. In facial cleansing, dissolved oxygen assists in exfoliating dead particles from the skin surface. Dissolved oxygen has also been used to remove toxins, particulates and other occlusions in skin pores. Further, oxygen may be able to oxidize oils in the skin pores, thus allowing the pores to be backfilled with water and become receptive to infiltration by beneficial lotions and other skin care products. Without the oxidative effects, pores in the skin would remain filled with oil that would require displacement from the pore before lotions could occupy pore volume.

Furthermore, oxygen has been used to revitalize skin cells by aiding in the production of collagen. For example, oxygen can revitalize skin cells by joining with protein molecules to nourish the cells and produce collagen. It is even possible that dissolved oxygen can stimulate hair follicles and consequentially hair growth.

Improvement in skin topography (roughness) has been also observed following exposure of the skin to oxygen dissolved in water. Stereoscopic examination of the skin indicates that the peaks that exist in the epidermal layer of the skin have been become smooth; presumably as a result of selectively higher oxidation rates associated with the higher surface area ridges of the skin.

Another use for a highly oxygenated solution is to oxygenate hemoglobin via the peripheral capillaries of humans and animals. Increasing blood oxygen content has many benefits including, but not limited to headache relief, improved circulation, and relief of muscle stiffness.

There are several accepted oxygen delivery methods, with hydrogen peroxide being a popular source of oxygen for topical applications and baths. Oxygen easily derives from peroxide, or H₂O₂, when a molecule of peroxide readily dissociates into water (H₂O) and an oxygen free-radical. That decomposition creates an enriched solution that facilitates dermal contact with oxygen. Hydrogen peroxide is distributed in various grades and concentrations that are specific to certain applications. Solutions of 3% and 6% hydrogen peroxide are commonly sold to consumers who use the solutions to disinfect cuts and clean skin areas. Solutions of 35% hydrogen peroxide are frequently added to spas and hot tubs to disinfect the water. Skin therapists also use the latter concentrations in oxygen baths to improve tissue regeneration and remove toxins from the dermis. Some topical creams contain stabilized forms of hydrogen peroxide for preventing free-radical formation and infections in skin.

The use of hydrogen peroxide for skin treatment is not without significant controversy. Some authorities claim that hydrogen peroxide is cytotoxic to human fibroblasts (due to the presence of free-radical oxygen). As a result, some medical professionals recommend additional dilution of peroxide solutions to avoid their toxic effects on skin. The literature also suggests that hydrogen peroxide reduces white blood cell activity. Still others have found that hydrogen peroxide slows wound healing by drying the wound, destroying the exudate and leading to some necrosis of skin tissue. Dry tissue also makes the wound area prone to bacterial growth and infection. As such, hydrogen peroxide has drawn questions as to its suitability for treating wounds and burns.

Even in the face of such controversy, preparations claiming to be oxygenated are available on the market today. Interestingly, oxygen is not often disclosed as an ingredient, and many of the ingredients that are disclosed have chemical incompatibility with oxygen. It has been shown through polargraphic dissolved oxygen or “DO” probes that very little if any of the oxygen claimed to be present actually remains dissolved in the marketed solution.

The amount of molecular oxygen dissolved into the matrix largely depends on the method used to dissolve or provide the gas during preparation. Two conditions must be satisfied: an oxygen source with sufficient potential to result in the desired oxygen concentration, and favorable reaction kinetics to allow the dissolution reaction to occur at a sufficient rate for the system to approach the equilibrium oxygen potential. Pressurized gas phase oxygen, oxygen derived from an in situ chemical reaction, and electrolytic derived oxygen would all be possible sources of oxygen. Similar processes can be used with other solute gases.

Favorable reaction kinetics requires the creation of interphase interfaces. These interfaces are necessary for the passage of a solute gas, such as oxygen, to occur into the matrix for dissolution or adsorption. If interface resistance is characteristically high, a large amount of interfacial area is needed for favorable kinetics. Additionally, diffusion limits liquid phase transport of most dissolved gases in water, and high interfacial area reduces the diffusion distance required by the solute.

The amount of oxygen initially dissolved into solution is largely dependent on the method used to dissolve the oxygen gas into solution. Generally, these methods consist of two steps: creating a solute gas/solvent liquid interfacial area, and, exposing the gas/liquid mixture to elevated pressure. The former step affects the kinetics or rate at which the solution process occurs while the latter determines the maximum theoretical dissolved. Small bubbles create interfacial area and promote more favorable kinetics. The second step is a pressure-concentration relationship, such as Henry's Law for dilute solutions and Sievert's Law for diatomic gases at higher concentrations. These steps may be combined, although the source of oxygen must operate at a higher final pressure rather than allowing a pump, for example, to pressurize both the liquid and gas components after the gas has been introduced.

One common method for oxygenating water is the coarse bubble aeration process, which is a subset of aeration methods known categorically as air diffusion. Pressurized air or oxygen gas is introduced through a submerged pipe having small holes or orifices into a container of water. Gas pressure is sufficient to overcome the hydrostatic head pressure, and also sustains pressure losses during passage through the small gas orifices. As a result, bubble aeration occurs at relatively low pressures; this pressure being predominantly a function of tube immersion depth and density of the liquid, which in this case is water.

Since all interphase interfaces have a characteristic surface energy, the creation of interfacial (surface) area is an energetic process. As a gas passes through an orifice, for example, pressure energy is converted to kinetic energy, which consequently satisfies the energetic requirements of the system for the production of surface area. Area and velocity are inversely proportional; hence, as the orifice diameter decreases, the corresponding pressure drop and gas velocity increase, and more surface area is generated. Smaller bubbles result. This process has a limiting condition, however, in that the amount of heat (as irreversible work) that is produced is inversely proportional to the square of orifice diameter. It therefore becomes impractical and energetically inefficient to operate at exceptionally small orifice diameters. This process also has an absolute limit as a gas velocity of Mach 1 is approached within the pore of a porous medium used to create bubbles. Because a pore lacks the convergent/divergent geometry required to achieve supersonic flow, increasing pressure beyond the critical pressure will not result in a further reduction of bubble size.

Since oxygen therefore is introduced into solution at relatively low pressures in the coarse bubble aeration process, the oxygen bubbles are relatively large. As a result, the aggregate bubble surface area for a dispersion of bubbles produced by bubble aeration is relatively small. The limited surface area produced by bubble aeration limits the rate at which gas can be dissolved and practically limits the concentration of gas that can be dissolved, into solution. Oxygen dissolution is a function of the interfacial contact area between gas bubbles and the surrounding medium, and bulk fluid transport (mixing) in the liquid phase. In particular, the rate of oxygen dissolution is directly proportional to the surface area of the bubbles. A dispersion of very small bubbles, e.g., bubbles having diameters in the order of 50 microns, will have a much larger total surface area than a, dispersion of large bubbles occupying the same volume. Consequently, the rate of oxygen dissolution in bubbling aeration is limited by the size of the bubbles introduced into the solvent. Fluid mixing is also very limited in bubbling aeration because the only energy source available for agitation is the isothermal expansion energy of oxygen as it rises in the solution.

Oxygen dissolution in bubbling aeration is also limited by ambient pressure conditions above the solution. If the solution being aerated is exposed to atmospheric conditions, the dissolved oxygen concentration will be limited to the solubility limit of oxygen (at its partial pressure in air of 0.21 atm.) under such conditions. The desirability of bubbling aeration is further hampered by equipment and energy requirements. Large blower units are used to force the gas bubbles into the carrying liquid. These blowers generate high-energy costs and often require special soundproof installations or other engineering costs. Bubble aeration is therefore an impractical process for producing oxygenated solutions or solution/suspensions for health related applications.

Other methodologies have been used to prepare oxygenated solutions based on pressure tanks and adaptations of carbonator devices that dissolve carbon dioxide in water. For a given pressure and temperature, the solubility of carbon dioxide in water exceeds that of oxygen by over an order of magnitude. Carbonators therefore are acceptable for preparing carbonated water solutions but not oxygenated solutions.

Accordingly, there exists a need in the art for gas transporting medium, and methodologies to create such medium. Further, there exists a need in the art for methodologies to operate at very high system pressures and also provide a means to impart high specific energy levels or power density to create surface area.

### SUMMARY OF THE INVENTION

A rheological medium is provided containing molecular oxygen and/or other gases of interest, such as nitrous oxide, at supersaturated concentrations above the equilibrium solubility limit at ambient conditions. This gasified rheological medium may supply a large amount of molecular oxygen, nitrous oxide, nitric oxide, carbon dioxide, and other gases in a manner that is not traumatic to skin tissue. Since the dissolution and adsorption of these gases occur during preparation under elevated pressure (hyperbaric conditions), a large concentration of gas may be dissolved into solution. Well known energetic barriers to bubble nucleation (“activation energy”) allow the solution to remain hyper-saturated when the solution is depressurized to ambient conditions. A hydrated clay component of the medium may also be included, and may adsorb gases. The resulting rheological medium may have an oxygen content, for example, of 8-70% by STP (Standard Temperature and Pressure) volume or about 860 mg/l. In one particular embodiment, the oxygen-enriched medium may be accompanied by a dispersion of micro-bubbles intentionally held in suspension by a Bingham plastic. These micro-bubbles subsequently spread and consolidate over a substrate, such as tissue, and from an intimate layer. The rheological properties of the matrix may allow the layer to remain intact. Silicone may be present to further enhance containment due to a characteristically high surface tension.

One method for using the oxygenated lotion/gel includes topically applying it to a user's wound area, such as a cut/laceration, superficial burn, or the like. Any layer thickness is envisioned to be within the scope of the invention, however thicker layers may provide the greatest concentration of dissolved gases and silicone to the skin. The lotion/gel may be allowed to enter tiny fissures or cavities in the user's skin tissue whereupon dissolved oxygen will be released for aiding in the regeneration of new tissue cells. Nitrous oxide, an analgesic, may also available to reduce pain as it is released from the lotion/gel. Mechanically emulsified silicone is present to add lubricity, facilitate scar reduction, and act as a barrier layer to topical contamination. The dispersion of gas micro-bubbles will be spread along with the lotion/gel and become a layer of film intimate with the skin. Due to its high surface tension, silicone helps to maintain the gas layer in contact with the skin, essentially emulating a gas chamber environment at atmospheric pressure.

### BRIEF DESCRIPTION OF THE DRAWINGS

Aspects, features, benefits and advantages of the embodiments herein will be apparent with regard to the following description, appended claims, and accompanying drawings. In the following figures, like numerals represent like features in the various views. It is to be noted that features and components in these drawings, illustrating the views of embodiments of the presently disclosed invention, unless stated to be otherwise, are not necessarily drawn to scale.

FIG. 1 is a flow chart showing the basic lotion/gel compositional components with optional additions shown by boxes with dotted line connectors;

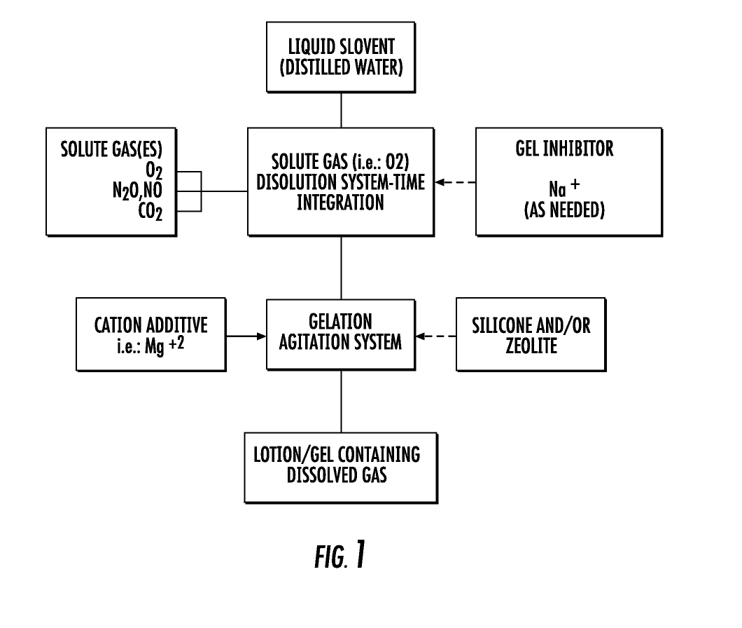

FIG. 2A is a flow chart showing the steps to one preferred method of lotion/gel manufacture with optional additions shown by boxes with dotted line connectors;

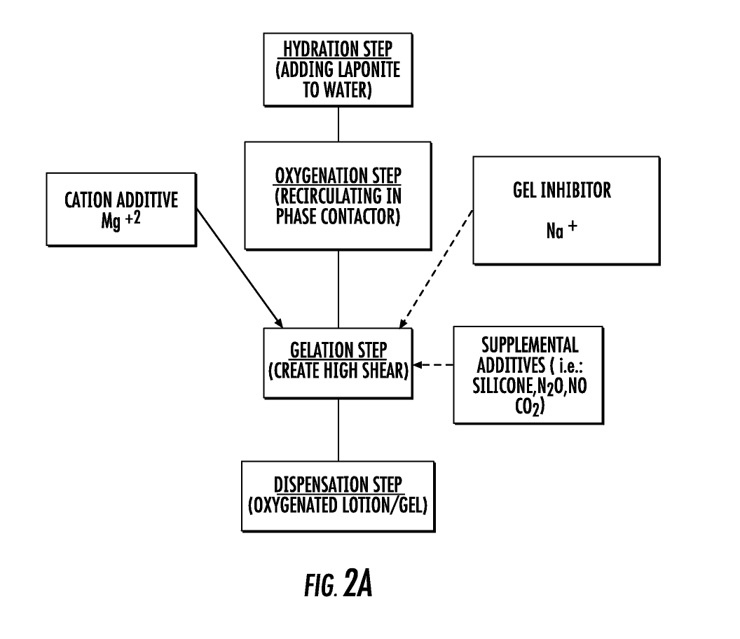

FIG. 2B is a flow chart depicting an alternate representation of said method;

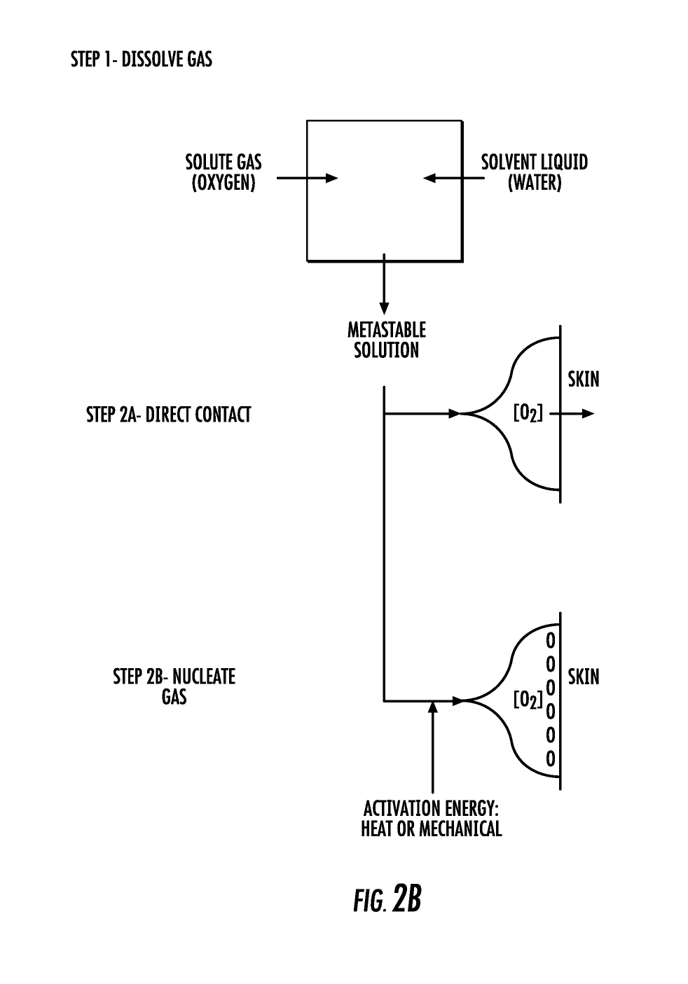

FIG. 3 is a photograph of one batch manufacturing equipment set up according to this invention;

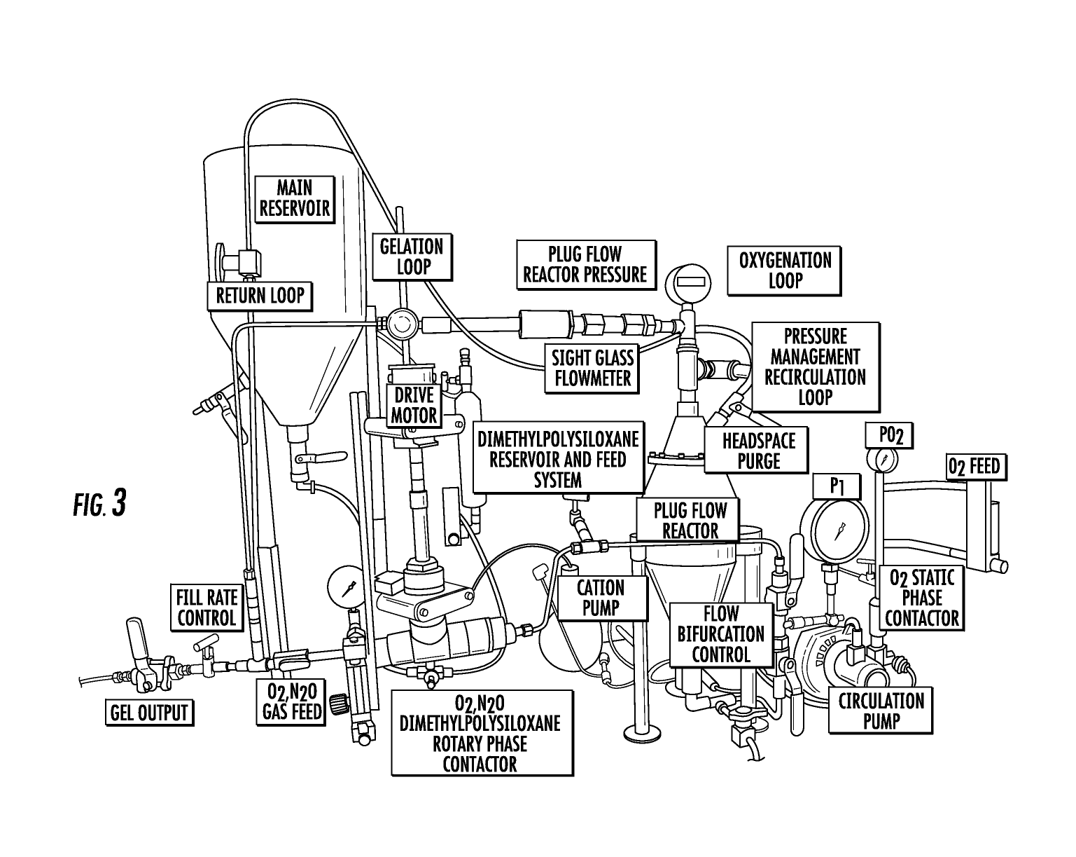

FIG. 4 is a schematic diagram showing a preferred representation of various method steps/equipment;

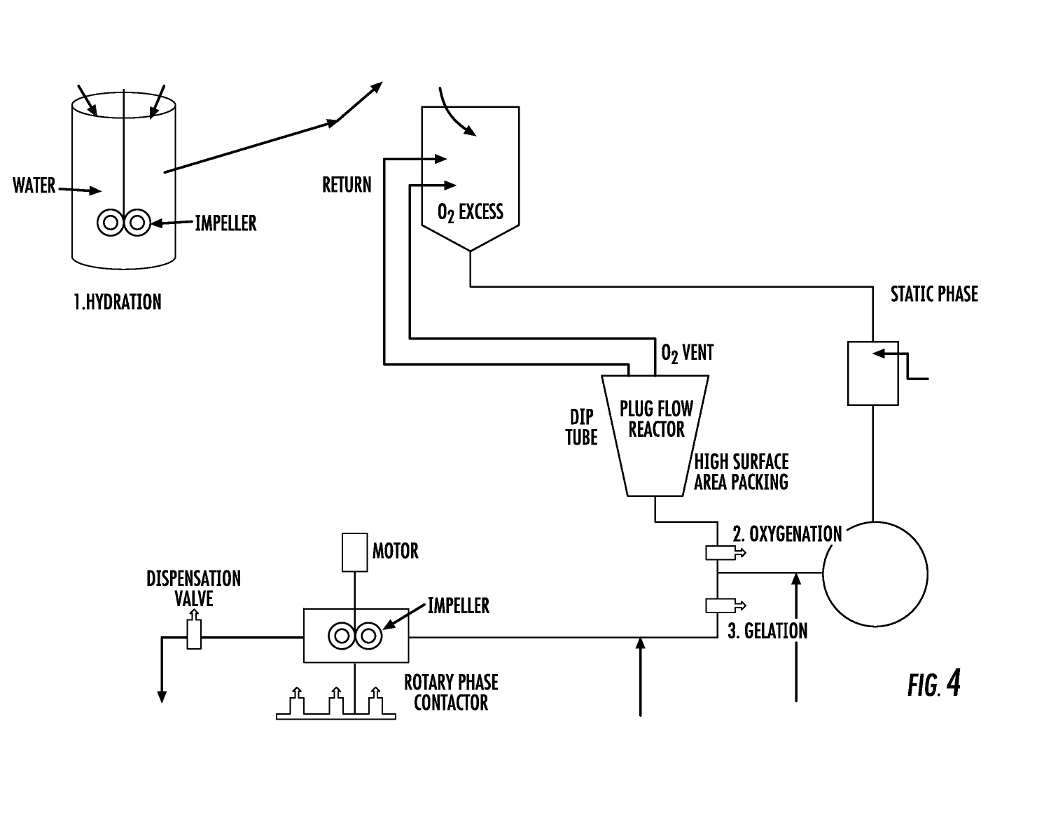

FIG. 5 is a photograph showing two side-by-side packets of a higher bubble version of this lotion/gel; and

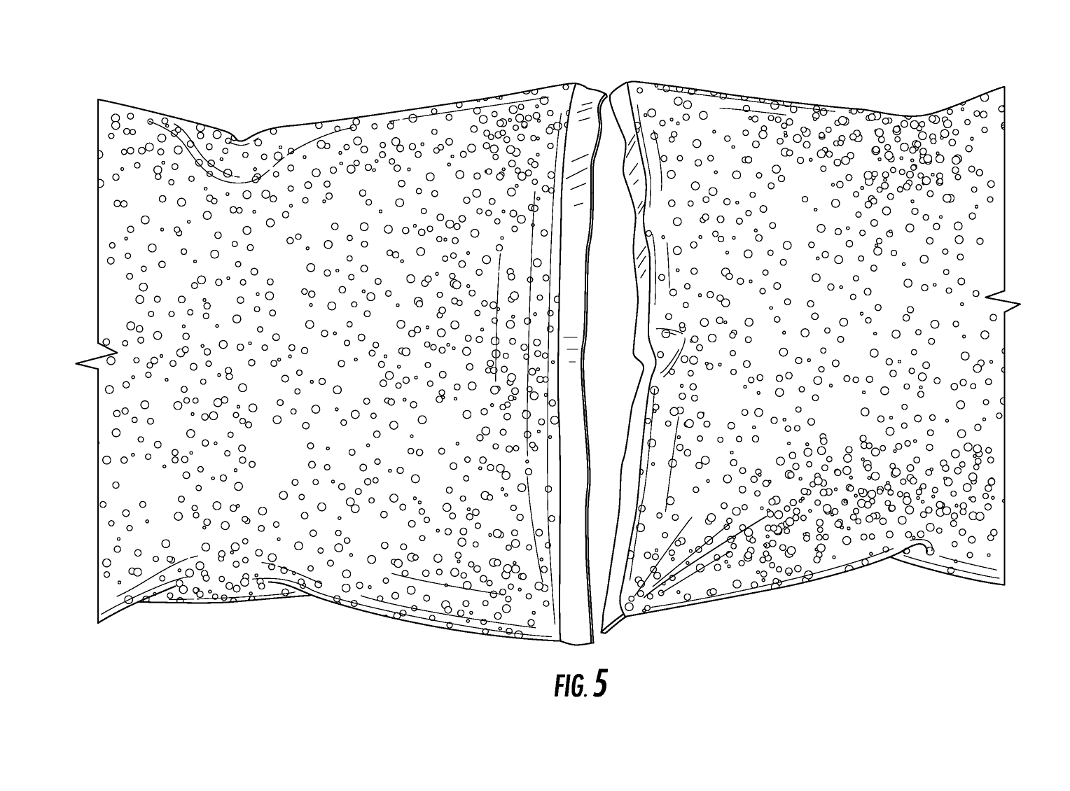

FIG. 6 is a table listing some known and other potential/possible end uses.

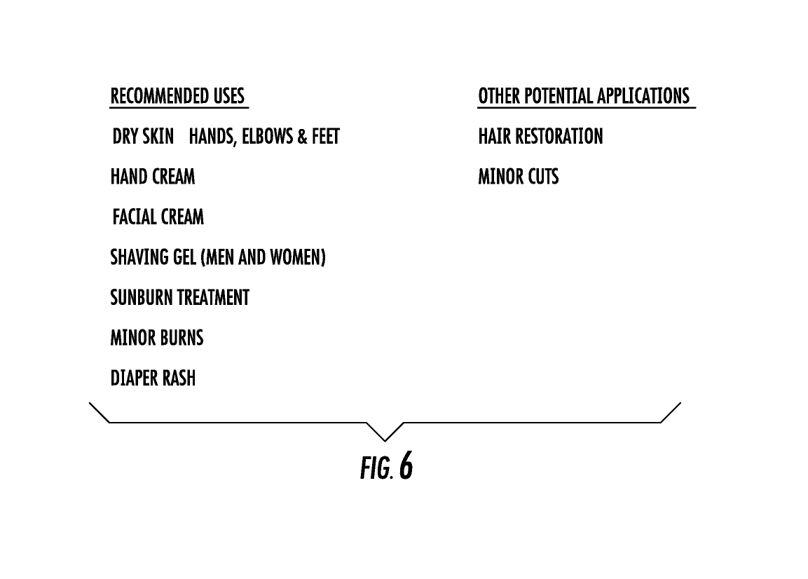

### DETAILED DESCRIPTION OF PREFERRED EMBODIMENTS

In the following description, the present invention is set forth in the context of various alternative embodiments and implementations involving a rheological medium containing molecular oxygen and/or other gases of interest, such as nitrous oxide, at supersaturated concentrations above the equilibrium solubility limit at ambient conditions. While the following description discloses numerous exemplary embodiments, the scope of the present patent application is not limited to the disclosed embodiments, but also encompasses combinations of the disclosed embodiments, as well as modifications to the disclosed embodiments.

Various aspects of the rheological medium may be illustrated with reference to one or more exemplary implementations. As used herein, the term “exemplary” means “serving as an example, instance, or illustration,” and should not necessarily be construed as preferred or advantageous over other variations of the devices, systems, or methods disclosed herein. “Optional” or “optionally” means that the subsequently described event or circumstance may or may not occur, and that the description includes instances where the event occurs and instances where it does not. In addition, the word “comprising” as used herein means “including, but not limited to”.

It must also be noted that as used herein and in the appended claims, the singular forms “a”, “an”, and “the” include the plural reference unless the context clearly dictates otherwise. Unless defined otherwise, all technical and scientific terms used herein have the same meanings as commonly understood by one of ordinary skill in the art.

The terms and expressions employed herein are used as terms of description and not of limitation. There is no intention in use of such terms and expressions of excluding any equivalents of the features shown and described or portions thereof. It is understood that various modifications of these embodiments are possible within the scope and spirit of the invention. While the two-phase oxygenated matrix has been described primarily in terms of its use in skin products and topical treatment, the invention is intended for use in any application where a supply of oxygen or another gas is desired. For example, the oxygenated solution could also be used to enhance tumor treatment or as an oxygen-enriched blood substitute. In the former case, oxygen delivered to the hypoxic area of a large tumor may increase the chemo-sensitivity or radio-sensitivity of tumor cells, allowing a malignant condition to be more amenable to treatment. As a blood substitute, the oxygen-enriched solution could possibly be administered intravenously in situations where whole blood products are not required. An example of such use is in response to blood loss due to hemorrhage, where fluid and oxygen are in critical need.

The invention is further intended to encompass a wide range of solutes and solvents other than oxygen and water. For instance, injecting nitrogen gas into a solvent can form a two-phase mixture in accord with the present invention. When using a lotion/gel matrix for skin debridement, a variety of gases may be added to the oxygen for safe tissue debriding including, but not limited to air, carbon dioxide or a number of inert gases. Gas may be dissolved into or even reacted with a number of different solvents, such as propylene glycol or perflubrons to form a two-phase mixture.

Solute gas(es) that have pharmaceutical benefit are of particular interest in the presently disclosed invention. While molecular oxygen, nitrous oxide, nitric oxide, carbon dioxide have been specifically mentioned, other gases having medical or pharmaceutical benefit are within the scope of the presently disclosed invention.

The matrix may contain a dispersion of gas bubbles and micro-bubbles in suspension. One such matrix contains oxygen and nitrous oxide dissolved in water and adsorbed into hydrated clay capable of cross linking to establish a Bingham Plastic matrix with the desired rheological properties. This example matrix may include suspended bubbles of oxygen and nitrous oxide in molecular form. Such bubbles are immobilized in the matrix provided that the buoyancy forces acting to gravimetrically separate the bubbles do not exceed the critical shear (flow) stress characteristic of the Bingham Plastic. It should be noted that the product will be referred to as a lotion/gel or as a matrix, and by matrix it is implied that we have a combination of solution, suspension, and adsorbed gas(es). Preferably, this matrix is in the form of a Bingham plastic, however, this characterization is not intended to limit the continuous phase to any particular rheology for the purposes of this invention.

When the matrix is spread on a surface, such as skin, the dissolved and adsorbed gas(es) are available for interaction with the skin, which may include trans-dermal transport of the gas(es). Additionally, the suspended gas bubbles will spread on the skin and establish a layer of the constitutive gas(es) at atmospheric pressure representing a chemical equivalent to placing skin in a chamber of the gas(es). Further, the dissolved and/or adsorbed gas(es) could represent an even higher chemical potential than a value corresponding to the pure gas at atmospheric pressure or even higher pressure, based on conditions used to prepare the medium. The well-known Henry's law establishes the relationship between medium preparation pressure and the resulting chemical potential of the dissolved gas.

A number of solute gases have medical applications. These include, but are not necessarily limited to: molecular oxygen, nitrous oxide, nitric oxide, and carbon dioxide. The most common solvent matrix with the greatest anticipated use is water. Saline, physiological pH buffered cell growth solution, water containing dissolved or suspended species that are compatible with the dissolved gas(es), and dimethyl sulfoxide (DMSO) are also candidate solvent matrices.

The lotion/gel matrix (hereinafter lotion/gel) of this invention contains exceptionally high levels of molecular oxygen. Unlike many lotions and gels on the market that purport to be oxygenated, the lotion/gel disclosed herein does not contain peroxides or ozone that form free radicals. The oxygen and nitrous oxide contained in this gel are both dissolved in the water solvent phase and adsorbed in a hydrated mineral. Further, visible gas bubbles are suspended as micro-bubbles that, in one preferred embodiment, are less than the diameter of most human hair. This lotion/gel, in one embodiment, may contain and retain almost 20 times the oxygen level found in tap water.

In a preferred embodiment, the lotion/gel contains 6 ingredients: aqua (purified water), Laponite (natural clay), oxygen, nitrous oxide, silicone (dimethylpolysiloxane), and magnesium cations in the form of Epson Salts (magnesium sulfate), compounded in a unique manner. Water, oxygen, and Laponite are natural ingredients. The resultant end product allows a user's skin to be exposed to oxygen levels previously unavailable for topical skin care. Molecular oxygen and nitrous oxide are both dissolved in the water phase and adsorbed by the clay at high levels. These combined gases are observed to promote healing, with nitrous oxide also functioning as a topical analgesic and exfoliant. Both gases are also available in a free gas form as suspended micro-bubbles, having a diameter less than 100 microns, and macro bubbles that have a size distribution of approximately 1 millimeter and larger. The Bingham plastic rheology of the lotion/gel may provide a stable suspension of gases. These gases may directly contact the skin as the gel is spread and represent what would be the equivalent to an oxygen “tent”, using pure gases at atmospheric pressure. This is advantageous as it is known in the art that oxygen enhances cellular metabolism and promotes tissue repair.

Silicone may desirably and significantly increase the surface tension of the lotion/gel, but is immiscible with the water base of the gel. Higher surface tension improves gas containment at the gel/skin interface. Furthermore, silicon has been used to reduce scar tissue development and facilitates the healing process. Incorporating chemical emulsifiers, surfactants, and/or co-solvents to promote silicone uptake and retention may adversely affect the healing impact of the lotion/gel. A very high energy density mechanical phase contactor has been developed as part of this invention to create a near emulsion of silicone in water without chemical emulsifiers.

A user group in excess of 300 individuals was created for a preferred embodiment of this invention lotion/gel; that is, an embodiment containing water, cross linked Laponite, oxygen, nitrous oxide, and silicone. Benefits in immediate pain relief and enhanced healing were reported by multiple individuals for the following conditions/uses: sunburn, thermal burns, post-surgical scar reduction, lacerations, acne, contact dermatitis (including poison ivy and diaper rash), deep tissue pain, age spot reduction, wrinkle abatement, joint pain, exfoliation, skin roughness, hemorrhoids, shaving, and baldness. Embodiments comprising higher levels of contained pharmaceutical gases and agents for possible regulated drug applications are also envisioned as within the scope of the presently disclosed invention.

A basic outline of various aspects of the presently disclosed invention is provided herein. Embodiments of the presently disclosed invention provide lotions/gels comprising gaseous species, including but not limited to oxygen, nitrous oxide, nitric oxide, carbon dioxide and the like which may be delivered to a target site in a liquid solvent. The stable hypersaturated gas/liquid solutions may be hypersaturated liquids with gas(es) of interest; may remain stable as a homogeneous liquid; and may be delivered to the target site by diffusion, which drives the transport of gas from solution to the target site (tissue or blood for example). The hypersaturated gas in liquid may be produced due to low diffusion of gas in liquids where a high gas/liquid surface area is created, preferably with agitation, which allows maximum transport with minimum flux (mass flow rate=flux×surface area). A thin film may develop that minimizes transport distances between the lotion/gel and target site.

A dispersed phase for additional gas storage is also envisioned as within the scope of the presently disclosed invention. Carbon dioxide has a high apparent solubility in water because carbonic acid forms that decomposes as carbon dioxide leaves solution. The decomposition of carbonic acid continues to supply carbon dioxide (and water) as the pressure over the liquid is reduced. This contributes markedly to the total quantity of gas that a given volume of liquid can evolve. No such chemical mechanism exists for oxygen in water, except for the decomposition of oxygen substitutes such as “Perflubron”. However, Perflubron and its decomposition products have a downside toxicity. A dispersed phase can be added to a gas saturated solvent (such as water) that adsorbs additional gas. This dispersed phase can either dissolve or remain as a physically distinct phase. Laponite is an example of a mineral that has been found to adsorb oxygen when exposed and subsequently desorb oxygen in the presence of a sink. In this manner, Laponite has a role similar to carbonic acid in the case of carbonated water.

Additional and/or selective gas storage is possible using zeolites. Zeolites are ceramics with controlled pore sizes. The pore radii can be selected on the basis of the size of the gas molecule intended to be adsorbed. Oxygen concentrators operating on the principle of pressure swing adsorption use zeolite columns. In the presently disclosed invention, a zeolite or zeolites of selected pore radii may be added to a Bingham plastic and immobilized as a uniform dispersion. Gases such as oxygen, nitric oxide, and nitrous oxide would be adsorbed into the zeolites during solubilization. The Bingham plastic characteristic of the gel would suspend the zeolites uniformly. Adsorbed gas(es) in the zeolite(s) would desorb upon contact of the gel with a sinking surface that will reduce the concentration of the gas in the gel phase.

In an augmented gel production process, 1.5-4.0% of a gelling agent may be added to the water and allowed to recirculate in the system to fully adsorb the gas of interest, i.e. oxygen. Once saturated, a quantity of oxygenated liquid may be shunted from the recirculation loop and into a shear inducing chamber while remaining at system pressure. Cations such as Mg⁺² may be added in the form of Epsom salts to facilitate gelling. The liquid that consists of water and the gelling agent saturated by the gas of interest (i.e. oxygen) begins to gel. Once fully gelled, the zeolite(s) may be added in the form of micron-sized powder. The gel is exposed to the additional gas (i.e., nitrous oxide) for the adsorption of the gas in the zeolite pores. Once the zeolite is fully saturated, the gel can be removed from the chamber (either continuously or in separate, distinct batches) for eventual consumer use.

One preferred micro-bubble dispersion consists primarily of oxygen gas bubbles for eventual nucleation from solution. An early micro-bubble suspension with a lower density than the solution phase had an occluded or cloudy appearance caused by the scattering of visible light energy through the smaller micro-bubble surfaces throughout. A subsequent variation having less oxygen but with larger bubbles for greater visual assurance to consumers as to the gaseous content so enclosed has been produced.

The solubility limit of oxygen in water under equilibrium conditions with air (pO₂=0.21) at 77° F. is approximately 8.3 mg/l. When a two-phase mixture is exposed to atmospheric conditions, the solution has a supersaturated oxygen content, i.e. above the solubility limit of oxygen in water under such conditions. Preferably, that homogeneous solution has a dissolved oxygen concentration above 20 mg/l at 1 atm and 65° F. More preferably, the solution 15 has a dissolved oxygen concentration above 40 mg/l at 1 atm and 65° F.

The supersaturated oxygen content in solution is preserved by storing it in a manner that limits or prevents gas desorption. For instance, the resultant lotion/gel may be stored and distributed in sealed screw top containers constructed of glass or alternative materials impervious to oxygen diffusion at these higher oxygen concentrations. If is stored in capped bottles made of an oxygen impervious material, elevated oxygen concentrations can be preserved for extended periods.

The micro-bubble dispersion is characterized as having a very large surface area through which interfacial transport of oxygen occurs. Interfacial transport of oxygen through a large surface area aids in resupplying oxygen to solution when dissolved oxygen is taken up during chemical reactions. As a result, a large surface area in the micro-bubble dispersion is desirable.

One representative matrix contains micro-bubbles having an average bubble diameter of about 10-100 microns. Micro-bubbles within this size range provide a significantly larger surface area than a cluster of large bubbles containing the same volume of gas. The magnitude of this difference can be visualized by performing calculations for several bubble diameters at a constant volume of gas. The following calculations show the surface areas present for a single bubble, a plurality of one-inch diameter bubbles and a plurality of 50-micron diameter bubbles, wherein each calculation is based on one cubic foot of gas.

The aggregate surface area for a dispersion of gas increases markedly as the radius of the bubbles decreases. For instance, a dispersion of 50-micron diameter bubbles containing one cubic foot of gas will have an aggregate surface area that is more than 500 times greater than a dispersion of one-inch bubbles containing the same volume of gas.

One novel aspect of this invention involves the substitution of a Newtonian solvent with a Bingham plastic. Such a material requires a finite yield stress to initiate movement. Applied stress levels that are below the yield stress threshold will not result in movement of the fluid. A Bingham plastic can be considered to have infinite viscosity and behave as a solid at stress levels below the yield stress. A Bingham plastic should result in bubble immobilization, provided that the magnitude of the buoyancy forces exerts a stress level that falls below the yield stress for the Bingham plastic. Bubble immobilization would thus provide stability of the micro-bubble suspension.

It has been discovered that the current invention produces stable suspensions of micro-bubbles when a Bingham plastic is used as the matrix. This is preferably accomplished by adding and mixing ingredients to form a Bingham plastic and oxygenated liquid at elevated pressure, i.e. prior to the formation of micro-bubbles. A mixer, that is u of the oxygenation process, can be used for this purpose. Since the components are mixed prior to the solution being reduced to ambient pressure, micro-bubbles will not substantially form. Once the solution is reduced in pressure, micro-bubbles will form. Such bubbles would be immobilized by the previously formed Bingham plastic, in any event.

A variety of Bingham plastics provide a suitable solvent phase, including but not limited to formulations using clay based thickening agents, such as Optigel-SH™ manufactured by Sud-Chemie, Inc., and formulations using polymeric based thickening agents, such as Carbopol™ polymers manufactured by B. F. Goodrich Company. Where oxygen micro-bubbles are used, Optigel-SH™ is a preferred solvent, because it contains an oxidation resistant substance. It has been found that oxygen micro-bubbles, immobilized in a Bingham plastic using a polymeric thickening agent, can react with the polymer and slowly release heat as a result of the reaction. The extended contact time provided by bubble immobilization allows this oxidation reaction to occur.

The Bingham plastic is characterized as having a finite yield stress. Fluid movement in a Bingham plastic will not occur until the finite yield stress is exceeded. Once the yield stress has been exceeded, the stress may increase linearly with increasing shear rate. Buoyancy forces acting on the oxygen micro-bubbles are insufficient to overcome the finite yield stress in the Bingham plastic. Therefore, the Bingham plastic immobilizes micro-bubbles in the mixture for extended periods.

As stated earlier, the two-phase micro-bubble containing oxygenated mixture can be used in any application in which oxygen is beneficial, including the treatment of skin wounds and burns. In one application, a skin wound may be treated topically with an oxygenated mixture to non-surgically remove dead, devitalized, contaminated and foreign matter from tissue cells.

The introduction of gas into a low-pressure stream creates a two-phase oxygenated mixture. That mixture, when conveyed through a pump known as a co-compressor increases the pressure of both the gas and liquid and discharges the mixture into a high-pressure discharge line. The pressure of gas and liquid are increased to allow large quantities of oxygen to efficiently dissolve in the liquid in a short period of time. The elevated pressure also substantially limits the remaining gas micro-bubbles from increasing in size. The amount of pressure in that line may vary depending on system size and desired discharge conditions. Preferably, the mixture pressure as it enters the discharge line is between about 150 and 800 psig. The dissolved oxygen content in the mixture at the point of discharge can be as high as 200 mg/l.

When applied, the matrix is allowed to enter tiny fissures or cavities in the wounded tissue. Some of the dissolved oxygen contacts the wounded tissue and aids in the regeneration of new tissue cells. As the matrix is circulated throughout the tissue layers, the dissolved oxygen nucleates into fine micro-bubbles that attach to skin fragments. These micro-bubbles exfoliate damaged tissue layers, assisting in their debridement and the regeneration of new tissue cells.

Energy may be added to the bath solution after the bath is filled to stimulate the nucleation of micro-bubbles and accelerate the exfoliation process. For instance, heat energy may be added to promote homogeneous nucleation. Mechanical mixing or circulation of the bath solution using stirring bars, circulation pumps or other mechanical devices may also stimulate nucleation of micro-bubbles. One preferred system employs a circulation pump to gently draw solution and recirculate it for some set time.

While specific embodiments of the invention have been described in detail, it should be appreciated by those skilled in the art that various modifications and alternations and applications could be developed in light of the overall teachings of the disclosure. Accordingly, the particular arrangements, systems, apparatuses, and methods disclosed are meant to be illustrative only and not limiting as to the scope of the invention.

What is claimed is:

1. A method for producing a gas transporting rheological medium, said method comprising:
(a) hydrating and dispersing a thickening agent in a fluid medium;
(b) adding a first gas for dissolution and/or adsorption the fluid medium using a minimum fluid system pressure of about 7 psig to the desired first gas concentration;
(c) mechanically emulsifying dimethylpolysiloxane into the fluid medium;
(d) adding a source of one or more +2 valence cations to the suspension for crosslinking to form a gel; and
(e) dispensing the gel into a container.

2. The method of claim 1, which further includes, after step (a):
adding one or more cation sources to the dispersed thickening agent for delaying a crosslinking reaction of the hydrated thickening agent in the fluid medium;

3. The method of claim 2 wherein the cation source is selected from the group consisting of: tetrasodium pyrophosphate, trisodium phosphate and combinations thereof.

4. The method of claim 3 wherein the cation source consists essentially of tetrasodium pyrophosphate.

5. The method of claim 1, which further includes, after step (b):
depressurizing the suspension to precipitate microbubbles of the first gas; and
re-pressurizing the suspension to add additional first gas.

6. The method of claim 1, which further includes, before step (c):
adsorbing additional first gas into the fluid.

7. The method of claim 1, which further includes, after step (d), at least one of the following additional sub-steps:
(i) reducing pressure of the gel to substantially atmospheric pressure for producing microbubbles; and
(ii) introducing additional solute gas to the gel for producing a suspension of macrobubbles.

8. The method of claim 1, which further includes, after step (d): introducing a second gas to the gel for producing a suspension of second gas bubbles.

9. The method of claim 8 wherein the second gas is selected from the group consisting of: nitrous oxide, carbon dioxide and mixtures thereof.

10. The method of claim 9 wherein the second gas consists essentially of nitrous oxide.

11. The method of claim 1 wherein step (e) further includes dispersing into the container with a substantially turbulent-free, sub-surface transfer for preserving the macrobubble and microbubble suspensions therein.

12. The method of claim 1 wherein the thickening agent of step (a) is selected from the group consisting of: laponite, bentonite, montmorillonite, magnesium aluminum silicate, sodium alginate, and various carbomers.

13. The method of claim 12 wherein the thickening agent of step (a) consists essentially of laponite.

14. The method of claim 1 wherein the first gas is selected from the group consisting of: oxygen, nitrous oxide, nitric oxide, carbon dioxide and mixtures thereof.

15. The method of claim 14 wherein the first gas consists essentially of oxygen.

16. The method of claim 1 wherein the +2 valence cation source of step (d) is selected from the group consisting of: magnesium sulfate, magnesium nitrate and combinations thereof.

17. The method of claim 16 wherein the +2 valence cation source consists essentially of magnesium sulfate.

18. A method for producing a gas transporting rheological medium, said method comprising:
(a) hydrating and dispersing a thickening agent in a suspension;
(b) adding a source of one or more +1 valence cations to the dispersed thickening agent for delaying a crosslinking reaction to the suspension;
(c) adsorbing a first solute gas in the suspension using a minimum system pressure of about 7 psig to a desired level of saturation for the first solute gas concentration;
(d) depressurizing the suspension to precipitate microbubbles of the first solute gas;
(e) re-pressurizing the suspension to add additional first solute gas, if desired;
(f) mechanically emulsifying dimethylpolysiloxane into the suspension with a rotary impeller phase contactor operating at a minimum viscosity of about 50-1000 centistokes and a minimum power density of about 8 w/l;
(g) adding a source of one or more +2 valence cations to the suspension for crosslinking to form a hydrated gel;
(h) reducing pressure of the hydrated gel to substantially atmospheric pressure for producing microbubbles; and
(i) dispensing the hydrated gel into a container with substantially turbulent-free sub-surface transfer while preserving the macrobubble and microbubble suspensions therein.

19. The method of claim 18 wherein the +1 valence cation source consists essentially of tetrasodium pyrophosphate and the +2 valence cation source consists essentially of magnesium sulfate.

20. The method of claim 18 wherein the first solute gas is selected from the group consisting of: oxygen, nitrous oxide, nitric oxide, carbon dioxide and mixtures thereof.
